SEQUENCE LISTING (1 sequence)

SEQ ID NO: 1 - protein, 314 aa, Homo sapiens
MAPPQVLAFGLLLAAATATFAAAQEECVCENYKLAVNCFVNNNRQCQCTSVGAQNTVICSKLAAKCLVMKAEMNGSKLGRRAKPEGALQNNDGLYDPDCDESGLFKAKQCNGTSMCWCVNTAGVRRTDKDTEITCSERVRTYWIIIELKHKAREKPYDSKSLRTALQKEITTRYQLDPKFITSILYENNVITIDLVQNSSQKTQNDVDIADVAYYFEKDVKGESLFHSKKMDLTVNGEQLDLDPGQTLIYYVDEKAPEFSMQGLKAGVIAVIVVVVIAVVAGIVVLVISRKKRMAKYEKAEIKEMGEMHRELNA

(19) United States
(12) Patent Application Publication
(10) Pub. No.: US 2016/0313335 A1
(43) Pub. Date: Oct. 27, 2016

(54) Methods for the Prognosis of Breast Cancer

(71) Applicants: Paul WALFISH, Toronto (CA); Ranju RALHAN, Thornhill (CA)

(72) Inventors: Paul WALFISH, Toronto (CA); Ranju RALHAN, Thornhill (CA)

(21) Appl. No.: 15/102,251

(22) PCT Filed: Dec. 5, 2014
(86) PCT No.: PCT/CA2014/051176

§ 371 Date: Jun. 6, 2016

Related U.S. Application Data
(63) Continuation of application No. 14/501,020, filed on Sep. 29, 2014.
Application No. 14/501,020 is a continuation-in-part of application No. 14/099,529, filed on Dec. 6, 2013, now abandoned.
Application No. 14/099,529 is a continuation-in-part of application No. 13/100,949, filed on May 4, 2011, now abandoned.
(60) Provisional application No. 61/330,966, filed on May 4, 2010.
(60) Provisional application No. 61/332,358, filed on May 7, 2010.

(51) Int. Cl. G01N 33/574

(52) U.S. Cl. CPC G01N 33/57415; G01N 2333/705

(57) ABSTRACT

Methods and kits for the prognosis of breast cancer comprising measurement of nuclear Ep-ICD poly-peptides are provided. Measurement may be quantitative and/or qualitative. The invention also provides a system for generating an Ep-ICD Subcellular Localization Index (ESLI) value, which may be used to prognose breast cancer in a subject.

### CROSS REFERENCE TO RELATED APPLICATIONS

This application claims priority under the Paris Convention from U.S. patent application Ser. No. 14/501,020, filed Sep. 29, 2014 and U.S. patent application Ser. No. 14/099,529, filed Dec. 6, 2013, each of which is incorporated herein by reference. This application is also a Continuation of U.S. patent application Ser. No. 14/501,020, filed Sep. 29, 2014, which is a Continuation in Part of U.S. patent application Ser. No. 14/099,529, filed Dec. 6, 2013, which is a Continuation in Part of U.S. patent application Ser. No. 13/100,949, filed May 4, 2011, which claims the benefit of priority under 35 U.S.C. §119(e) to U.S. Patent Application No. 61/330,966, filed May 4, 2010 and U.S. Provisional Patent Application No. 61/332,358, filed May 7, 2010. Each of the aforementioned applications is incorporated by reference herein as if set forth in its entirety.

### FIELD OF THE INVENTION

The present description relates generally to the field of prognosing cancer. More particularly, the description relates to methods and kits for prognosing breast cancer.

### BACKGROUND OF THE INVENTION

Breast cancer is the most frequently diagnosed cancer in females, with an estimated 1.38 million new cases per year worldwide and an estimated 226,870 new cases in the United States in 2012 (Siegel et al., CA Cancer J. Clin. 2012, 62(1):10-29; Ferlay et al., Int. J Cancer 2010, 127(12):2893-2917). In early stage breast carcinoma patients, the presence of metastases to axillary lymph nodes is thought to be the most important predictor of survival (Fitzgibbons et al., Arch Pathol Lab Med 2000, 124(7):966-978). Patients with node-positive tumors have up to an 8-fold increase in mortality relative to node-negative patients (Arriagada et al., Cancer 2006, 106(4):743-750). Population breast cancer screening with mammography has been promoted to facilitate early detection of breast tumors and it may have the potential to lower mortality, but it is also associated with unnecessary treatment of tumors that may not have adversely affected the patient (e.g., non-aggressive tumors) (Gotzsche & Jorgensen, Cochrane Database Syst Rev 2013, 6:CD001877).

Current clinical therapies for breast cancer include surgery, radiotherapy and drug therapies targeting oncogenic processes. Prediction of patient response to therapy and propensity for metastasis in patients is challenging, at least in part due to an incomplete understanding of the biology of various breast cancer subtypes. Many patients are over-treated to improve overall survival rates in early breast cancer. Defining individual risk of disease recurrence and/or individual sensitivity to treatment might reduce over-treatment. Genomic tests (Mammaprint, Oncotype Dx, PAM50) and immunohistochemical tests (IHC 4) have been developed for prediction of breast cancer prognosis and response to chemotherapy but prospective validation of these tests is not currently available (Azim et al., Annals of Oncology 2013, 24(3):647-654). Nuclear magnetic resonance (NMR) and mass spectrometry (MS)-based serum metabolite profiling has been shown to accurately identify 80% of breast cancer patients whose tumors failed to respond to chemotherapy (Wei et al., Molecular oncology 2013, 7(3):297-307). A five-gene Integrated Cytokine score (ICS) has been proposed for predicting metastatic outcome from primary HRneg/Tneg breast tumors independent of nodal status, adjuvant chemotherapy use, and Tneg molecular subtype (Yau et al., Breast Cancer Research 2013, 15(5):R103).

Epithelial cell adhesion molecule (EpCAM) has been widely explored as an epithelial cancer antigen (Munz et al. 2009, Cancer Res 69: 5627-5629). EpCAM is a glycosylated, 30- to 40-kDa type I membrane protein, expressed in several human epithelial tissues, and overexpressed in some cancers as well as in some progenitor and stem cells (Munz et al. 2009, Mukherjee et al. 2009; Am J Pathol 175: 2277-2287; Carpenter & Red Brewer 2009, Cancer Cell 15: 165-166; Schnell et al. 2013, Biochim Biophys Acta 1828: 1989-2001; Ni et al. 2012, Cancer Metastasis Rev 31: 779-791). EpCAM is comprised of an extracellular domain (EpEx) with epidermal growth factor (EGF) and thyroglobulin repeat-like domains, a single transmembrane domain, and a 26-amino acid intracellular domain called Ep-ICD. In normal cells, the full length EpCAM protein is sequestered in tight junctions and therefore not easily accessible to antibodies. In cancer cells, EpCAM is homogeneously distributed on the surface of cancer cells. EpCAM has been explored as a surface-binding site for therapeutic antibodies.

EpCAM is expressed in a majority of human epithelial cancers, including breast, colon, gastric, head and neck, prostate, pancreas, ovarian and lung cancer and is one of the most widely investigated proteins for its diagnostic and therapeutic potential (Spizzo et al. 2004, Breast Cancer Res Treat 86: 207-213; Went et al. 2004, Hum Pathol 35: 122-128; Saadatmand et al. 2013, Br J Surg 100: 252-260; Soysal et al. 2013, Br J Cancer 108: 1480-1487). An EpCAM expression-based assay is the only FDA-approved test widely used to detect circulating tumor cells in breast cancer patients (Cristofanilli et al. 2004, N Engl J Med 351: 781-791).

EpCAM-targeted molecular therapies are being studied for several cancers including breast, ovarian, gastric and lung cancer (Baeuerle & Gires 2007, Br. J Cancer 96: 417-423; Simon et al. 2013, Expert Opin Drug Deliv 10: 451-468). EpCAM expression has been used to predict response to anti-EpCAM antibodies in breast cancer patients (Baeuerle & Gires 2007, Schmidt et al. 2005, Annals of Oncology 23: 2306-2313; Schmidt et al. 2010, Annals of Oncology 21: 275-282). Clinical trials of anti-EpCAM antibodies targeting the EpEx domain have shown limited efficacy in cancer therapy and the prognostic potential for EpCAM in determining survival of cancer patients remains unclear (Riethmuller et al. 1998, J Clin Oncol 16: 1788-1794; Fields et al. 2009, J Clin Oncol 27: 1941-1947; Gires & Bauerle et al. 2010, J Clin Oncol 28: e239-240; author reply e241-232; Schmoll & Arnold 2009, J Clin Oncol 27: 1926-1929; Maetzel et al. 2009, Nat Cell Biol 11: 162-171). For example, increased EpCAM expression has been associated with a favorable prognosis in colorectal and gastric cancers (Songun et al. 2005, Br J Cancer 92:1767-1772; Went et al. 2006, Br J Cancer 94:128-135; Ensinger et al. 2006, J Immunother 29:569-573; Ralhan et al. 2010, BMC Cancer 10:331). In contrast, it has been suggested that increased EpCAM expression is a marker of poor prognosis in breast and gall bladder cancers (Gastl et al. 2000, Lancet 356:1981-1982; Varga et al. 2004, Clin Cancer Res 10:3131-3136).

The paradoxical association of EpCAM expression with prognosis in different cancers may be explained by functional studies of EpCAM biology using in vitro and in vivo cancer models (van der Gun et al. 2010, Carcinogenesis 31: 1913-1921), and the recently unraveled mode of activation of EpCAM oncogenic signaling by proteolysis, and the potential of Ep-ICD in triggering more aggressive oncogenesis (Maetzel et al. Nat. Cell Biol. 2009, 11:162-171). Regulated intra-membrane proteolysis of EpCAM results in shedding of EpEx and release of Ep-ICD into the cytoplasm, nuclear translocation and activation of oncogenic signaling (Carpenter & Brewer, Cancer Cell, 2009, 15:156-166). Subcellular localization of the EpEx and Ep-ICD fragments of EpCAM was not considered in the above study linking EpCAM overexpression to prognosis of breast cancer (Gastl et al., 2000).

The present inventors earlier reported on Ep-ICD and EpEx expression analyses and their potential for use as diagnostic markers in ten epithelial cancers, including breast cancer (US Patent Publication No. 2011/0275530). With respect to diagnosis of breast cancer, the presence of nuclear Ep-ICD was found to be a marker of cancerous breast tissue relative to non-cancerous breast tissue (US Patent Publication No. 2011/0275530).

Methods and kits for use in prognosis of breast cancer are desirable.

### SUMMARY OF THE DISCLOSURE

In a first aspect, the disclosed invention provides a method for prognosing breast cancer in a subject. The method comprises: (a) measuring an amount of nuclear Ep-ICD in a biological sample from the subject; (b) comparing the amount measured in the biological sample to a control; and prognosing breast cancer based on the comparison between the measured amount of nuclear Ep-ICD and the control.

In one embodiment of the first aspect, if the control is: an amount of nuclear Ep-ICD in a non-aggressive breast cancer sample, then a higher measured amount of nuclear Ep-ICD indicates a poor prognosis, and an equal or lower measured amount of nuclear Ep-ICD indicates a favorable prognosis; or an amount of nuclear Ep-ICD in an aggressive breast cancer sample, then an equal or higher measured amount of nuclear Ep-ICD indicates a poor prognosis.

In one preferred embodiment, the non-aggressive breast cancer sample is known not to progress in disease for at least 40 months following measurement of the nuclear Ep-ICD amount. In one preferred embodiment the aggressive breast cancer sample is known to progress in disease in less than about five years following measurement of the nuclear Ep-ICD amount. In one preferred embodiment, the poor prognosis comprises disease free survival of less than five years. In one preferred embodiment, the disease free survival is less than or equal to about 41 months. In one preferred embodiment, the favorable prognosis comprises disease free survival of at least about five years.

In one embodiment of the first aspect, the biological sample from the subject is obtained post-therapeutic treatment. In one preferred embodiment, the biological sample from the subject comprises one or more of breast epithelial cells, breast tissue, breast tumor tissue, and stage I or II breast cancer tumor cells.

In one embodiment of the first aspect, the breast cancer prognosed is invasive ductal carcinoma, invasive lobular carcinoma, invasive mucinous carcinoma, ductal carcinoma in situ, or lobular carcinoma in situ.

In one embodiment of the first aspect, the measured amount of nuclear Ep-ICD is one or more of a quantitative and qualitative amount. In one preferred embodiment, the quantitative amount is a percentage of cells in the biological sample that are positive for nuclear Ep-ICD or an absolute quantity of nuclear Ep-ICD. In one preferred embodiment, the qualitative amount is an intensity of signal emitted by a label indicative of nuclear Ep-ICD.

In one embodiment of the first aspect, the method further comprises determining quantitative and qualitative scores for nuclear Ep-ICD and cytoplasmic Ep-ICD, wherein increased quantitative and qualitative nuclear and cytoplasmic Ep-ICD scores are associated with a poor prognosis of breast cancer.

In one preferred embodiment of the first aspect, the determining of the quantitative and qualitative nuclear Ep-ICD and cytoplasmic Ep-ICD scores comprises: (i) contacting the sample with: a binding agent that specifically binds to Ep-ICD or part thereof and a detectable label for detecting binding of the first binding agent to Ep-ICD, wherein the detectable label emits a detectable signal upon binding of the binding agent to Ep-ICD; (ii)

measuring: (a) a first percentage, comprising the percentage of cells in the sample having Ep-ICD in the nucleus bound to the binding agent, and assigning a first quantitative score to the first percentage according to a first scale; and (b) a second percentage, comprising the percentage of cells in the sample having Ep-ICD in the cytoplasm bound to the binding agent, and assigning a second quantitative score to the second percentage according to the first scale; (iii) measuring: (a) a first intensity, comprising the intensity of the signal emitted in the nucleus by the label, and assigning a first qualitative score to the first intensity according to a second scale; and (b) a second intensity, comprising the intensity of the signal emitted in the cytoplasm by the label and assigning a second qualitative score to the second intensity according to the second scale.

In one preferred embodiment of the first aspect, the method further comprises calculating total nuclear Ep-ICD and cytoplasmic Ep-ICD scores, the calculating comprising: (a) adding the first quantitative and qualitative scores to generate the total nuclear Ep-ICD score; and (b) adding the second quantitative and qualitative scores to generate the total cytoplasmic Ep-ICD score.

In one preferred embodiment of the first aspect, the method further comprises: calculating an Ep-ICD Subcellular Localization Index (ESLI) value for the sample, the ESLI value being a sum of the total nuclear Ep-ICD score and the total cytoplasmic Ep-ICD score, divided by two; comparing the calculated ESLI value to a reference value, wherein the reference value is: (i) an ESLI value indicative of a non-aggressive breast cancer; or (ii) an ESLI value indicative of an aggressive breast cancer; and determining a poor prognosis of breast cancer in the subject when the calculated ESLI value of the sample is greater than the reference value of (i) or is greater than or equal to the reference value of (ii).

In one preferred embodiment of the first aspect, the binding agent is an antibody. In one preferred embodiment, the label is chosen from detectable radioisotopes, luminescent compounds, fluorescent compounds, enzymatic labels, biotinyl groups and predetermined polypeptide epitopes recognizable by a secondary reporter.

In one preferred embodiment of the first aspect, the quantitative amount is obtained using immunohistochemical (IHC) analysis. In one preferred embodiment, the qualitative amount is obtained using immunohistochemical (IHC) analysis.

In one preferred embodiment of the first aspect, the first scale comprises the following scores: a score of 0 is assigned when less than 10% of the cells are positive; a score of 1 is assigned when 10-30% of the cells are positive; a score of 2 is assigned when 31-50% the cells are positive; a score of 3 is assigned when 51-70% of the cells are positive; and a score of 4 is assigned when more than 70% of the cells are positive, and the second scale comprises the following scores: a score of 0 is assigned when no signal is detected; a score of 1 is assigned when a mild signal is detected; a score of 2 is assigned when a moderate signal is detected; and a score of 3 is assigned when an intense signal is detected. In one preferred embodiment, an ESLI value indicative of non-aggressive breast cancer is less than 3 and an ESLI value indicative of aggressive breast cancer is greater than or equal to 3.

In one preferred embodiment of the first aspect, the measuring of an amount of nuclear Ep-ICD is manual or automated.

Other objects, features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### DESCRIPTION OF THE DRAWINGS

The invention will now be described in relation to the drawings in which:

FIGS. 1A and 1B depict immunohistochemical analysis of Ep-ICD and EpEx expression in breast cancer. FIG. 1A depicts representative photomicrographs demonstrating: (I) predominantly cytoplasmic Ep-ICD expression in normal breast tissues; nuclear and cytoplasmic accumulation of Ep-ICD in (II) DCIS; (Ill) IDC; (IV) ILC; (V) IMC; and (VI) negative control breast cancer tissue incubated with isotype specific IgG showing no detectable immunostaining for Ep-ICD. FIG. 1B depicts expression of EpEx in: (I) normal breast tissues; (II) DCIS; (III) IDC; (IV) ILC; and (V) IMC. Original magnification ×400; arrows labelled N, C and M depict nuclear, cytoplasmic and membrane staining, respectively.

FIGS. 2A and 2B depict Kaplan-Meier curves for disease-free survival (DFS) stratified by nuclear Ep-ICD expression in all breast carcinoma patients and in IDC patients, respectively. FIG. 2A shows nuclear accumulation of Ep-ICD was associated with significantly reduced DFS in the entire cohort of breast carcinoma patients (p<0.001). FIG. 2B shows nuclear accumulation of Ep-ICD was associated with significantly reduced DFS in IDC patients (p<0.001).

FIGS. 3A and 3B show Ep-ICD Subcellular Localization Index (ESLI) values and disease free survival in breast cancer patients and IDC patients, respectively.

### DETAILED DESCRIPTION OF THE DISCLOSURE

Unless defined otherwise, all technical and scientific terms used herein have the same meaning as commonly understood by one of ordinary skill in the art to which this invention belongs.

### DEFINITIONS

The term “EpCAM” as used herein, refers to the epithelial cell adhesion molecule having the amino acid sequence set forth in SEQ ID NO: 1 (SEQ ID NO: 1 corresponds to Genbank Accession No. NP_002345). EpCAM comprises an extracellular domain, referred to herein as “EpEx”, that is 265 amino acids in length (amino acids 1-265 in SEQ ID NO: 1), a single transmembrane domain that is 23 amino acids in length (amino acids 266-288 in SEQ ID NO. 1), and an intracellular domain, referred to herein as “Ep-ICD”, that is 26 amino acids in length (amino acids 289-314 in SEQ ID NO. 1).

The term “aggressive” as used herein, refers to a type of cancer that forms, grows and/or spreads more quickly than a “non-aggressive” cancer. For example, a subject having an aggressive breast cancer may have an expected disease free survival (DFS) time that is less than a subject having a non-aggressive breast cancer. The DFS is the time period until disease recurrence, metastasis and/or death.

The term “score” as used herein, refers to a rating or grade provided to a result, wherein the rating or grade is measured on a scale that comprises minimum and maximum possible scores for a result.

The terms “algorithm” and “ESLI algorithm” as used herein, refer to a mathematical formula for numerically characterizing Ep-ICD sub-cellular expression by determining a value (i.e., an Ep-ICD Subcellular Localization Index “ESLI” value). The algorithm is defined further herein.

“Prognosis”, as used herein, refers to a prediction of the probable course and/or outcome of a disease. For example, a poor prognosis may predict a reduced DFS in a patient relative to a patient having a good prognosis. For example, a poor prognosis would predict a DFS of less than about five years and a favourable or good prognosis would predict a DFS of more than about five years.

As described herein, the inventors have found that breast cancer patients having a poor prognosis have breast tissue comprising an increased amount of Ep-ICD, in particular increased nuclear Ep-ICD, relative to breast cancer patients having a favorable prognosis. Methods prognosing breast cancer comprising one or more of detecting, measuring, scoring and evaluating subcellular localization of Ep-ICD are discussed further below. In one aspect, the invention provides a numerical scoring method to quantify prognosis, such scoring method is referred to herein as the Ep-ICD Subcellular Localization Index (ESLI). The use of an ESLI value in prognosing breast cancer is discussed further below.

Methods for the Prognosis of Breast Cancer

The present disclosure is generally directed to a method for prognosing cancer, in particular breast cancer, in a subject. The subject, also referred to herein as a patient, may be a mammal that is afflicted with, suspected of having, at risk for pre-disposal to, or being screened for breast cancer. In a preferred embodiment, the subject is a human.

In one embodiment, an amount of nuclear and/or cytoplasmic Ep-ICD is measured in a biological sample from the subject. The biological sample comprises breast epithelial cells. In a preferred embodiment, the biological sample comprises breast tissue. In a particularly preferred embodiment the biological sample comprises breast cancer tumor cells, such as, for example, stage I and/or II breast cancer tumor cells.

Measurement of Ep-ICD may be quantitative and/or qualitative. In one embodiment, measurement may be achieved by contacting the biological sample with a first binding agent and measuring in one or more nuclei and/or cytoplasms of the biological sample the amount of the first binding agent bound to Ep-ICD. In one embodiment, an amount of membranous EpEx is measured in a biological sample from the subject. Measurement of EpEx may be achieved by contacting the biological sample with a second binding agent and measuring in one or more membranes of the biological sample the amount of the second binding agent bound to EpEx. A binding agent refers to a substance that specifically binds to a specific polypeptide. A binding agent may be, for example, an antibody, a ribosome, RNA, DNA, a polypeptide or an aptamer. For example, an antibody specifically reactive with Ep-ICD may be used to detect Ep-ICD in the biological sample and may be used to determine the subcellular localization of Ep-ICD (i.e., nuclear or cytoplasmic). General techniques for in vitro detection of antigens in samples are well known in the art. In a preferred embodiment, an Ep-ICD-specific antibody is used to detect Ep-ICD. In a preferred embodiment, an EpEx-specific antibody is used to detect EpEx.

Binding agents specific for Ep-ICD or EpEx may be labelled with a detectable substance which facilitates identification in biological samples based upon the presence of the detectable substance. Examples of detectable substances include, but are not limited to, the following: radioisotopes, fluorescent labels, luminescent labels, bioluminescent labels, enzymatic labels, biotinyl groups, and predetermined polypeptide epitopes recognized by a secondary reporter. Binding agents may also be coupled to electron dense substances, such as ferritin or colloidal gold, which are readily visualized by electron microscopy.

Indirect methods may also be employed in which a primary antigen-antibody reaction is amplified by the introduction of a second antibody, having specificity for the antibody reactive against an epitope of the target polypeptide. For example, if the antibody having specificity against an Ep-ICD polypeptide is a rabbit IgG antibody, the second antibody may be goat anti-rabbit IgG, Fc fragment specific antibody labelled with a detectable substance, as described herein.

Methods for conjugating or labelling the antibodies discussed above may be readily accomplished by one of ordinary skill in the art.

Quantitative and/or qualitative measurement of Ep-ICD and/or EpEx may be automated or it may be done manually.

In one embodiment, quantitative and/or qualitative measurement of Ep-ICD may be automated using software, such as, for example, Visiopharm™ software. For example, the inventors have scanned IHC-treated breast cancer tissue samples using a NanoZoomer at 20× magnification. The scanned Images were loaded onto the Visiopharm Integrator System (VIS, version 4.6.3.857; Visiopharm, Hoersholm, Denmark) for digital analysis. Regions-of-interest (ROI) were manually drawn on each digital image. Regions within the ROIs were analyzed by the VIS to measure 3,3′-Diaminobenzidine (DAB) staining in epithelial cells in the nuclei, cytoplasm and/or membrane and to measure the intensity of staining. Results of this analysis were then used to stratify patients based on their risk for disease reoccurrence.

One example of manual quantitative and qualitative measurement of Ep-ICD, wherein scores are assigned to nuclear and cytoplasmic Ep-ICD quantitative and qualitative measurements, is described further below.

In one embodiment, once an amount of nuclear Ep-ICD is measured in a biological sample from the subject, the measured amount is compared to a control and a poor or favorable prognosis is made based on results of the comparison.

In one embodiment, the control is an amount of nuclear Ep-ICD in a non-aggressive cancerous biological sample, for example, a non-aggressive cancerous breast tissue or a sample comprising non-aggressive cancerous breast epithelial cells. In this case, a higher detected amount of nuclear Ep-ICD in the biological sample relative to the control indicates a poor prognosis of breast cancer and an equal or lower detected amount of nuclear Ep-ICD in the biological sample relative to the control indicates a favorable prognosis. For example, in one preferred embodiment, the control is the amount of nuclear Ep-ICD in a biological sample known not to progress to breast cancer for at least 40 months following measurement of the control amount. In this embodiment, a higher detected amount of nuclear Ep-ICD in the biological sample relative to the control indicates a poor prognosis, and an equal or lower detected amount of nuclear Ep-ICD in the biological sample relative to the control indicates a favorable prognosis.

In one embodiment, the control is an amount of nuclear Ep-ICD in an aggressive cancerous biological sample, for example, an aggressive breast tumor or a sample comprising aggressive cancerous breast epithelial cells. In this case, an equal or higher detected amount of nuclear Ep-ICD in the biological sample relative to the control indicates a poor prognosis of breast cancer. For example, in one preferred embodiment, the control is the amount of nuclear Ep-ICD in a biological sample known to progress to breast cancer in less than about five years following measurement of the control amount. In this embodiment, an equal or higher detected amount of nuclear Ep-ICD in the biological sample relative to the control indicates a poor prognosis.

In one embodiment, the breast cancer prognosed using the method provided herein is invasive ductal carcinoma (IDC), invasive lobular carcinoma (ILC), invasive mucinous carcinoma (IMC), ductal carcinoma in situ (DCIS) or lobular carcinoma in situ (LCIS).

In one embodiment, a method for prognosing breast cancer in a subject is provided, wherein the method comprises determining quantitative and qualitative scores corresponding to the amounts of nuclear Ep-ICD and cytoplasmic Ep-ICD. In this method, the quantitative and qualitative nuclear and cytoplasmic Ep-ICD scores are calculated and compared to control values for determining the poor prognosis of breast cancer in a subject.

In an aspect of the embodiment, the method may further comprise a step of calculating an Ep-ICD Subcellular Localization Index (ESLI) value for a sample obtained from the subject. The ESLI value, as discussed further below, offers a unique quantitative means of prognosing breast cancer in a subject.

The present inventors developed the ESLI algorithm by: i) examining subcellular localization of Ep-ICD in samples from subjects having healthy breasts and various stages of breast cancer; ii) determining associations between Ep-ICD subcellular localization and DFS times in breast cancer patients; iii) determining that both quantitative and qualitative measurement of subcellular localization of Ep-ICD provided useful prognostic information; iv) generating an algorithm for using the quantitative and qualitative data to calculate a value with prognostic significance; and v) generating scales and equations for use in the algorithm, wherein the scales are appropriate for scoring the quantitative and qualitative data and weighting the quantitative and qualitative data with respect to one another. In a particularly preferred embodiment, the combination of collecting quantitative and qualitative data regarding Ep-ICD subcellular localization in a breast tissue sample, applying the ESLI algorithm to the collected data to generate and ESLI value for the sample, comparing the ESLI value of the sample to a reference value facilitates prognosis of prognosis of breast cancer in subjects. In a particularly preferred embodiment, the quantitative and qualitative data are collected from tissue samples prepared for IHC.

Details of the ESLI breast cancer prognosis method and the ESLI algorithm are discussed further below.

In order to calculate an ESLI value, quantitative and qualitative nuclear Ep-ICD and cytoplasmic Ep-ICD scores are determined for a breast tissue sample obtained from a subject. The breast tissue sample would comprise cells (e.g., epithelial cells), each of such cells having a nucleus and cytoplasm.

In one embodiment, determination of quantitative and qualitative nuclear Ep-ICD and cytoplasmic Ep-ICD scores is done manually and comprises the following four steps.

(i) The sample is contacted with a binding agent that specifically binds to Ep-ICD or part thereof. A detectable label is used to detect binding of the binding agent to Ep-ICD. As discussed above, the detectable label may, for example, emit a detectable signal upon binding of the binding agent to Ep-ICD. In one aspect, the binding agent may be a labelled antibody specific to Ep-ICD. The label may be chosen from, for example, detectable radioisotopes, luminescent compounds, fluorescent compounds, enzymatic labels, biotinyl groups and predetermined polypeptide epitopes recognizable by a secondary reporter.

(ii) Subcellular localization of Ep-ICD is measured quantitatively and scored based on the percentages of cells in a tissue sample that are positive for Ep-ICD in (a) their nucleus and (b) their cytoplasm. The percentage of cells in a tissue sample that are positive for nuclear Ep-ICD expression is referred to as the “first percentage”. The first percentage is then assigned a score according to a scale that correlates percentage ranges with integer values. Such score and scale are referred to as the “first quantitative score” and “first scale”. The percentage of cells in a measured tissue sample that are positive for cytoplasmic Ep-ICD expression is referred to as the “second percentage”. The second percentage is then assigned a “second quantitative score” according to the first scale.

In one aspect, the first percentage (i.e., the percentage positive for nuclear Ep-ICD) and the second percentage (i.e., the percentage positive for cytoplasmic Ep-ICD) are scored according to the following first scale: when less than 10% of cells are positive a score of 0 is assigned; when 10-30% cells are positive a score of 1 is assigned; when 31-50% cells are positive a score of 2 is assigned; when 51-70% of cells are positive a score of 3 is assigned; and when more than 70% of cells are positive a score of 4 is assigned. It will be understood that such numerical scale is used only for convenience and is provided as an example. Various other scaling methods can also be used.

In one embodiment, the first and second percentages are obtained from tissue samples prepared for IHC. Immunohistochemistry (IHC) is a known method for demonstrating the presence and location of one or more specific proteins in tissue sections. Briefly, IHC comprises fixing and embedding a tissue sample, sectioning the tissue, mounting the tissue section, deparaffinizing and rehydrating the section, antigen retrieval, immunohistochemical staining, optional counterstaining, dehydrating and stabilizing with mounting medium, and viewing the stained section under a microscope.

In one embodiment, wherein the first and second percentages are obtained using IHC, a cell that is positive for nuclear and/or cytoplasmic Ep-ICD is one that is immunopositive (i.e., a cell comprising staining or fluorescence that is detectable upon microscopic examination and indicative of the Ep-ICD-specific antibody used in IHC of the sample).

(iii) Subcellular localization of Ep-ICD is measured qualitatively and scored based on the intensity of the signals emitted by a detectable label of an Ep-ICD binding agent in (a) the nucleus and (b) the cytoplasm of cells in the tissue sample. The intensity of the signal detected in the nucleus of the cells in the tissue is referred to as the “first intensity”. The first intensity is then assigned a score according to a scale that correlates a categorical assessment of signal intensity (e.g., categories ranging from zero detectable signal to a maximum or near maximum detected signal) with integer values. Such score and scale are referred to as the “first qualitative score” and “second scale”. The intensity of the signal detected in the cytoplasm of the cells in the tissue is referred to as the “second intensity”. The second intensity is then assigned a “second qualitative” score according to the second scale.

In one aspect, the first intensity (i.e., the categorical assessment of nuclear Ep-ICD binding agent signal emission) and the second intensity (i.e., the categorical assessment of cytoplasmic Ep-ICD binding agent signal emission) are scored according to the following second scale: when no signal is detected a score of 0 is assigned; when a mild signal is detected a score of 1 is assigned; when a moderate signal is detected a score of 2 is assigned; and when an intense signal is detected a score of 3 is assigned. Various other scaling methods may also be used.

In one embodiment, the first and second intensities are obtained using IHC analysis. In one preferred embodiment, the antibody-antigen interaction (i.e., the anti-Ep-ICD-Ep-ICD interaction) is visualized using chromogenic detection, in which an enzyme conjugated to the antibody cleaves a substrate to produce a colored precipitate at the location of the protein. In another preferred embodiment, the antibody-antigen interaction is visualized using fluorescent detection, in which a fluorophore is conjugated to the antibody and the location of the fluorophore can be visualized using fluorescence microscopy.

(iv) A total nuclear Ep-ICD score and a total cytoplasmic Ep-ICD score are calculated by adding the first quantitative and qualitative scores to generate the total nuclear Ep-ICD score and adding the second quantitative and qualitative scores to generate the total cytoplasmic Ep-ICD score.

In one preferred embodiment, after determining nuclear Ep-ICD and cytoplasmic Ep-ICD scores, an Ep-ICD Subcellular Localization Index (ESLI) value for the sample is calculated. In one example, the ESLI value is the sum of the total nuclear Ep-ICD score and the total cytoplasmic Ep-ICD score. In one preferred embodiment, the ESLI value is the sum of the nuclear Ep-ICD score and the cytoplasmic Ep-ICD score, divided by two (such arithmetic function being for convenience).

The calculated ESLI value is then compared to a reference value in order to determine a prognosis of breast cancer in the subject. The reference value is a predetermined cut-off value, wherein values on one side of the cut off value indicate a poor prognosis of breast cancer and vales on the other side of the cut-off value indicate a favourable prognosis of breast cancer.

In one embodiment, the reference value is an ESLI value indicative of a non-aggressive cancerous breast tissue. In this embodiment, a poor prognosis of breast cancer in the subject is determined when the calculated ESLI value of the sample is greater than the reference value. In this embodiment, a favourable prognosis of breast cancer is determined when the calculated ESLI value of the sample is less than or equal to the reference value.

In one embodiment, the reference value is an ESLI value indicative of an aggressive breast cancer. For example, the sample may be obtained from an aggressive breast tumour tissue. In this embodiment, a poor prognosis of breast cancer in the subject is determined when the calculated ESLI value of the sample is greater than or equal to the reference value.

In a preferred embodiment, the reference value is determined by retrospectively analyzing a plurality of breast cancer patients' tissue samples and corresponding patient clinical data regarding time of DFS.

In a particularly preferred embodiment, wherein an ESLI value is calculated using total nuclear and cytoplasmic Ep-ICD scores generated according to the aforementioned first and second scales (i.e., 0-4 and 0-3 for percentage positivity and intensity, respectively), a finding of an ESLI value of greater than or equal to 3 is indicative of aggressive breast cancer and a poor prognosis of breast cancer.

In one embodiment, a method for detecting abnormal subcellular localization of Ep-ICD in a breast tissue sample obtained from a subject is provided. In an aspect, the method comprises measuring an amount of nuclear Ep-ICD in a biological sample from the subject, comparing the amount detected in the biological sample to a control; and detection of abnormal subcellular localization of Ep-ICD in the breast the breast tissue sample is made based on the comparison between the detected amount of nuclear Ep-ICD and the control. Measurement may be quantitative and/or qualitative, as described herein. The control may be a non-aggressive or aggressive breast cancer, as described herein. Detection of an abnormal subcellular localization of Ep-ICD in a breast tissue sample is found when the measured amount of Ep-ICD is greater than that of the non-aggressive control or greater than or equal to that of the aggressive control.

In another embodiment, the method for detecting abnormal subcellular localization of Ep-ICD in a breast tissue sample obtained from a subject comprises the steps of (A) measuring nuclear and cytoplasmic Ep-ICD scores for the sample, (B) calculating an ESLI value for the sample and (C) comparing the calculated ESLI value to a reference value. The measuring and calculating steps may be carried out as discussed above with respect to breast cancer prognosis. In this embodiment, abnormal subcellular localization of Ep-ICD in the breast tissue sample is detected when the calculated ESLI value of the sample is greater than a reference value corresponding to an ESLI value indicative of a non-aggressive cancerous breast tissue; or when the calculated ESLI value of the sample is greater than or equal to a reference value corresponding to an ESLI value indicative of an aggressive breast cancer.

In the above description, scoring of Ep-ICD amounts was described in terms of a visual, i.e., manual, method. However, as will be understood, an automated method may also be used, such as the method using Visiopharm software, described above.

Kits

The present disclosure contemplates kits for carrying out the methods disclosed herein. Such kits typically comprise two or more components required for performing a prognostic breast cancer assay. Components include but are not limited to one or more of compounds, reagents, containers, equipment and instructions for using the kit. Accordingly, the methods described herein may be performed by utilizing pre-packaged prognostic kits provided herein. In one embodiment, the kit comprises one or more of binding agents, standards, stains, fixatives and instructions. In some embodiments, the instructions comprise one or more reference values for use as controls.

In one embodiment, the kit comprises one or more binding agents as described herein for prognosing breast cancer. By way of example, the kit may contain antibodies specific for Ep-ICD, antibodies against the Ep-ICD antibodies labelled with an enzyme(s), and a substrate for the enzyme(s). The kit may further contain antibodies specific for EpEX, antibodies against the EpEX antibodies labelled with an enzyme(s), and a substrate for the enzyme(s). The kit may also contain one or more of microtiter plates, reagents (e.g., standards, buffers), adhesive plate covers, and instructions for carrying out a method using the kit.

In one embodiment, the kit comprises antibodies or antibody fragments which bind specifically to epitopes of Ep-ICD and means for detecting binding of the antibodies to their epitopes associated with breast cancer cells, either as concentrates (including lyophilized compositions), which may be further diluted prior to testing. For example, a kit for prognosing breast cancer may contain a known amount of a first binding agent that specifically binds to Ep-ICD, wherein the first specific binding agent comprises a detectable substance or has the capacity to bind directly or indirectly to a detectable substance. In one embodiment, the kit further comprises antibodies or antibody fragments which bind specifically to epitopes of EpEX and means for detecting binding of the EpEX-specific antibodies to their epitopes associated with breast cancer cells, either as concentrates (including lyophilized compositions), which may be further diluted prior to testing.

In one embodiment, the kit comprises one or more binding agents, standards, stains, fixatives and instructions for measuring nuclear Ep-ICD and optionally membrane EpEX. For example, a kit comprising such binding agents, standards, stains fixatives and instructions may be used to practice methods disclosed herein. In a preferred embodiment, the kit can be used to practice a method disclosed herein that comprises IHC.

In one embodiment, the kit may further comprise tools useful for collecting biological samples (e.g. a breast tissue sample).

The following non-limiting example illustrative of the disclosure is provided.

### Example

The prognostic utility of subcellular Ep-ICD expression and membranous EpEx expression in breast cancer are examined. Correlation of subcellular Ep-ICD and membranous EpEX expression with clinic-pathological parameters and follow up of breast cancer patients are also examined.

Methods

This retrospective study of biomarkers using breast cancer patients' tissue blocks stored in the archives of the Department of Pathology and Laboratory Medicine and their anonymized clinical data was approved by the Mount Sinai Hospital Research Ethics Board, Toronto, Canada.

Patient and Tumor Specimens

The patient cohort consists of 266 breast cancer patients treated at Mount Sinai Hospital (MSH) between 2000 and 2007. The cohort consists of patients who had mastectomy or lumpectomy.

Inclusion criteria: Breast cancer tissue samples of patients who had up to 60 months follow-up with or without an adverse clinical event; availability of clinical, pathological and treatment data in the clinical database.

Exclusion criteria: Breast cancer tissues were not considered for this study if patient follow-up data were not available in the clinical database.

Normal breast tissues were chosen from breast reduction surgeries, normal tissue with adjacent benign lesions, and prophylactic mastectomies. Normal breast tissues from adjacent cancers were not included in this study. The patient cohort consisted of individuals with invasive ductal carcinoma (IDC) (n=180), invasive lobular carcinoma (ILC) (n=15), invasive mucinous carcinoma (IMC) (n=9), ductal carcinoma in situ (DCIS) (n=61), lobular carcinoma in situ (LCIS) (n=1), and 45 individuals with normal breast tissues. Breast cancer diagnosis was based on histopathological analysis of patient tissue specimens. The follow-up time for all patients including IDC cases in the study was 60 months. The clinicopathological parameters recorded included age at surgery, tumor histotype, tumor size, AJCC pTNM stage, nodal status, tumor grade, recurrence of disease, ER/PR status, hormonal treatment, radiation therapy, and/or chemotherapy. Formalin-fixed paraffin-embedded tissue blocks of all patients included in this study were retrieved from the MSH tumor bank, reviewed by the pathologists and used for cutting tissue sections for immunohistochemical staining with Ep-ICD and EpEx specific antibodies, as described below.

Immunohistochemistry (IHC)

Formalin-fixed paraffin embedded sections (4 μm thickness) of breast carcinomas were used for Ep-ICD and EpEx immunostaining, as described previously (Ralhan et al., BMC Cancer 2010, 10(1):331). In brief, for EpEx following deparaffinization and rehydration, antigen retrieval was carried out using a microwave oven in 0.01 M citrate buffer, pH 3.0 and endogenous peroxidase activity was blocked by incubating the tissue sections in hydrogen peroxide (0.3%, v/v) for 20 min. For Ep-ICD, the tissue sections were de-paraffinized by baking at 62° C. for 1 hour in vertical orientation, treated with xylene and graded alcohol series, and the non-specific binding was blocked with normal horse or goat serum. Rabbit anti-human Ep-ICD monoclonal antibody from Epitomics Inc. (Burlingame, Calif.) was used in this study. The α-Ep-ICD antibody 1144 recognizes the cytoplasmic domain of human EpCAM and has been used in our previous study of Ep-ICD expression in thyroid carcinoma and other epithelial cancers [Ralhan et al., BMC Cancer 2010]. Anti-EpCAM monoclonal antibody EpEx (MOC-31, AbD Serotec, Oxford, UK) recognizes an extracellular component (EGF1 domain-aa 27-59) in the amino-terminal region (Chaudry et al., Br J Cancer 2007, 96(7):1013-1019). The sections were incubated with either α-Ep-ICD rabbit monoclonal antibody 1144 (dilution 1:1500) or mouse monoclonal antibody MOC-31 (dilution 1:200) for 60 minutes, followed by biotinylated secondary antibody (goat anti-rabbit or goat anti-mouse) for 20 minutes. The sections were finally incubated with VECTASTAIN Elite ABC Reagent (Vector Laboratories, Burlington, ON, Canada) and diaminobenzidine was used as the chromogen. Tissue sections were then counterstained with hematoxylin. Negative controls comprised of breast tissue sections incubated with isotype specific IgG in place of the primary antibody, and positive controls (colon cancer tissue sections known to express Ep-ICD) were included with each batch of staining for both Ep-ICD and EpEx.

Evaluation of IHC and Scoring

Immunopositive staining was manually evaluated in the five most pathologically aggressive areas of the tissue sections by two researchers blinded to the final outcome and the average of these five scores was calculated as previously described (Ralhan et al., BMC Cancer 2010). Sections were scored on the basis of both the percentage of immunopositive cells and intensity of staining.

For percentage positivity, cells were assigned scores based on the following scale: 0, <10% cells; 1, 10-30% cells; 2, 31-50% cells; 3, 51-70% cells; and 4, >70% cells showing immunoreactivity.

Sections were also scored qualitatively on the basis of intensity of staining as follows: 0, none; 1, mild; 2, moderate; and 3, intense.

A total score (ranging from 0 to 7) for each tissue section was obtained by adding the scores of percentage positivity and intensity for each of the breast cancer tissue sections. The average total score from the five areas was used for further statistical analysis. Each tissue section was scored for cytoplasmic and nuclear Ep-ICD as well as for membrane EpEx following the aforementioned percentage positivity and intensity scales.

Statistical Analysis of IHC Data

The IHC data were subjected to statistical analysis with SPSS 21.0 software (SPSS, Chicago, Ill.) and GraphPad Prism 6.02 software (GraphPad Software, La Jolla, Calif.) as described previously (Ralhan et al., Mol Cell Proteomics 2008, 7(6):1162-1173]. A two-tailed p-value was obtained in all analyses and a p value<0.05 was considered statistically significant. Chi-square analysis was used to determine the relationship between Ep-ICD and EpEx expression and the clinicopathological parameters. Disease-free survival (DFS) was analyzed by the Kaplan-Meier method and multivariate Cox regression. Hazard ratios (HR), 95% confidence intervals (95% CI), and p values were estimated using the log-rank test. Disease-free survival or adverse clinical event (defined as clinical recurrence, distal metastases, and/or death) was considered to be the endpoint of the study. The cut-offs for IHC statistical analysis were based upon the optimal sensitivity and specificity obtained from the Receiver operating curves as described before (Ralhan et al., PLoS One 2010, 5(11):e14130). For nuclear Ep-ICD, IHC scores of ≧2 were considered immunopositive for all tissues analyzed. Ep-ICD cytoplasmic IHC scores of ≧4 were considered immunopositive for all tissues analyzed. Membranous EpEx IHC scores of ≧3 were considered immunopositive for all tissues analyzed.

Ep-ICD Subcellular Localization Index (ESLI) Scoring

Following evaluation and scoring of the IHC data, a calculation was made of the ESLI. The ESLI was calculated according to the following equation: ESLI=½×(% positivity score of nuclear Ep-ICD+intensity score of nuclear Ep-ICD+% positivity score of cytoplasm Ep-ICD+intensity score of cytoplasm Ep-ICD). As indicated above, the % positivity score comprises a score on a scale of 0 to 4 and the intensity score comprises a score on a scale of 0 to 3. An ESLI cutoff value of 3 was found to be useful for distinguishing between samples from patients having good and poor prognoses. For example, an ESLI value of ≧3 was considered a “positive” result and indicative of a poor breast cancer prognosis and an ESLI value of <3 was considered a “negative” result and indicative of a good prognosis of breast cancer.

Results

The clinicopathological parameters and treatment details of 266 breast carcinomas, including 180 IDC cases and 45 normal controls are summarized in Table 1. The median age of patients was 59.9 years (range 30.6-89.8 years). AJCC pTNM Stage I (35.3%) and II (32.7%) comprised a large proportion of tumors in this cohort. Tumor grades distribution was Grade I—21.1%; II—39.8%, and III—32.0%. Among the IDC cases, majority were also AJCC pTNM Stage I (62.8%) and II (32.2%). The IDC cases comprised of Grade I—23.3%; Grade II—36.7%; and Grade III—36.1% tumors.

TABLE 1
Clinicopathological characteristics of breast cancer patients.

| | Breast Cancer | | IDC | |
|---|---|---|---|---|
| | (n = 266) | | (n = 180) | |
| Surgical Treatment | | | | |
| Lumpectomy | 168 | (63.1%) | 113 | (62.8%) |
| Mastectomy | 84 | (31.6%) | 59 | (32.8%) |
| Unknown | 14 | (5.3%) | 8 | (4.4%) |
| Age at diagnosis (years) | | | | |
| Median (Range - 30.6-89.8) | 59.2 | | 59.2 | |
| <59 yrs | 126 | (47.4%) | 88 | (48.9%) |
| ≧59 yrs | 140 | (52.6) | 92 | (51.1%) |
| Adjuvant treatment | | | | |
| Hormonal treatment | | | | |
| Tamoxifen | 131 | (49.2%) | 94 | (52.2%) |
| Aromatase Inhibitor | 13 | (4.9%) | 8 | (4.4%) |
| Chemotherapy | 73 | (2.7%) | 66 | (24.8%) |
| Radiotherapy | 149 | (56.0%) | 101 | (56.1%) |
| Therapy details not available | 51 | (19.1%) | 30 | (16.6%) |
| Tumor size (cm) | | | | |
| Mean ± SD | 1.85 ± 1.525 | | 1.82 ± 1.466 | |
| Minimum | 0.1 | | 0.1 | |
| Maximum | 9 | | 9 | |
| ≦2 cm | 198 | | 81 | |
| >2 cm | 57 | | 96 | |
| Unknown | 11 | | 3 | |
| AJCC pTNM Stage (n, %) | | | | |
| 0 (DCIS + LCIS) | 62 | (23.3%) | — | |
| I | 94 | (35.3%) | 113 | (62.8%) |
| II | 87 | (32.7%) | 58 | (32.2%) |
| III | 6 | (2.3%) | 5 | (2.8%) |
| IV | 17 | (6.4%) | 4 | (2.2%) |
| Estrogen receptor (ER) | | | | |
| Negative | 35 | (13.1%) | 33 | (18.3%) |
| Positive | 161 | (60.6%) | 136 | (75.6%) |
| Unknown | 70 | (26.3%) | 11 | (6.1%) |
| Progesterone receptor (PR) | | | | |
| Negative | 71 | (26.7%) | 64 | (35.6%) |
| Positive | 123 | (46.2%) | 103 | (57.2%) |
| Unknown | 72 | (27.1%) | 13 | (7.2%) |
| Grade | | | | |
| I | 56 | (21.1%) | 42 | (23.3%) |
| II | 106 | (39.8%) | 66 | (36.7%) |
| III | 85 | (32.0%) | 65 | (36.1%) |
| Unknown | 19 | (7.1%) | 7 | (3.9%) |
| Nodal status | | | | |
| Negative | 204 | (76.7%) | 123 | (68.3%) |
| Positive | 62 | (23.3%) | 57 | (31.7%) |

Expression of Ep-ICD and EpEx in Breast Cancer Tissues

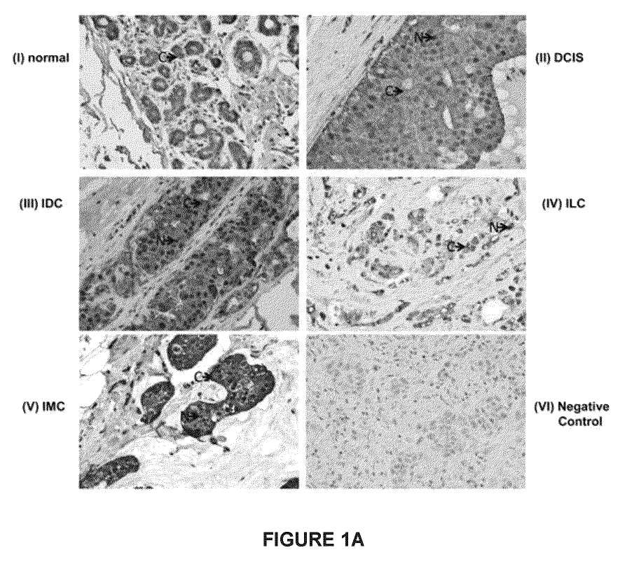

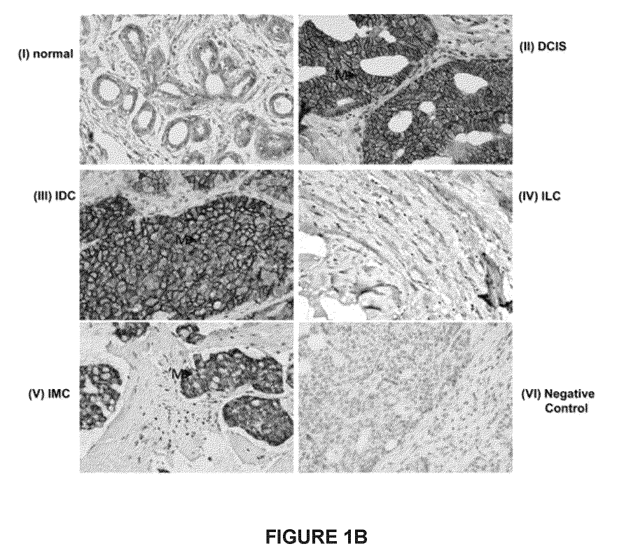

To determine the pattern of expression of Ep-ICD and EpEx in breast cancer histotypes, tissues of DCIS, IDC, ILC, and IMC were analyzed by IHC and compared to normal (i.e., non-cancerous) breast tissues. A summary of the percentage positivity for nuclear Ep-ICD, cytoplasmic Ep-ICD, and membranous EpEx and loss of membranous EpEx is provided in Table 2. Representative photomicrographs of Ep-ICD and EpEx expression in breast cancer subtypes are shown in FIGS. 1(A and B). Of 266 breast carcinomas examined, 121 (46%) were positive for nuclear Ep-ICD and 185 (70%) were positive for membranous EpEx, while 81 cases showed loss of membranous EpEx expression. This compares to 11 of 45 (24%) normal breast tissues immunopositive for nuclear Ep-ICD and 19 of 45 (42%) positive for membranous EpEx. Notably, 12 of 15 (80%) ILCs showed loss of membranous EpEx, compared to 14 of 61 (23%) DCIS, 52 of 180 (29%) IDC, and 3 of 9 (33%) IMC. Cytoplasmic Ep-ICD was frequently present in all histologic subtypes examined and normal tissues (87% normal tissues, 79% DCIS, 81% IDC, 80% ILC, and 100% IMC). Nuclear Ep-ICD was more frequently positive in breast carcinomas (121 of 266, 46%) compared to normal tissues (11 of 45, 24%). Evaluation of the individual subtypes showed nuclear Ep-ICD accumulation was frequently detected in ILC (10 of 15 tumors, 67%), 30 of 61 (49%) DCIS, 75 of 180 (42%) IDC, and 5 of 9 (56%) IMC cases.

TABLE 2
Expression of nuclear and cytoplasmic Ep-ICD and membranous EpEX
in normal tissues and breast cancer tissues having various histotypes (for nuclear Ep-ICD a
cut off IHC score of ≧2 was used to determine positivity, for cytoplasmic Ep-ICD a cut off
IHC score of ≧4 was used to determine positivity, for membranous EpEx a cut off IHC score
of ≧3 was used to determine positivity; “*” is used to note that one LCIS histotype sample
was included in the study, but LCIS data are not shown in the table).

| | | | Nuclear | | Cytoplasmic | | Membranous | | | |
|---|---|---|---|---|---|---|---|---|---|---|
| | | | Ep-ICD | | Ep-ICD | | EpEx | | Loss of membranous | |
| | Number of | | Positivity | | Positivity | | Positivity | | EpEx | |
| | Tissues N | | n (%) | | n (%) | | n (%) | | n (%) | |
| Tissue type | | | | | | | | | | |
| Normal | 45 | | 11 | (24%) | 39 | (87%) | 19 | (42%) | 26 | (58%) |
| Breast | 266 | | 121 | (46%) | 215 | (81%) | 185 | (70%) | 81 | (30%) |
| Cancer | | | | | | | | | | |
| Histotypes* | | | | | | | | | | |
| DCIS | 61 | (22.9%) | 30 | (49%) | 48 | (79%) | 47 | (77%) | 14 | (23%) |
| IDC | 180 | (67.6%) | 75 | (42%) | 145 | (81%) | 128 | (71%) | 52 | (29%) |
| ILC | 15 | (5.6%) | 10 | (67%) | 12 | (80%) | 3 | (20%) | 12 | (80%) |
| IMC | 9 | (3.4%) | 5 | (56%) | 9 | (100%) | 6 | (67%) | 3 | (33%) |

Relationship of Ep-ICD with Clinicopathological Characteristics of IDC Patients.

Nuclear and cytoplasmic Ep-ICD expression in IDC patients and their association with the clinicopathological characteristics are provided in Table 3. Nuclear Ep-ICD accumulation was significantly associated with, and observed in, all IDC patients with clinical recurrences (25 of 25 patients, 100%; p<0.001, Odds ratio (OR)=1.50, 95% confidence interval (CI)=1.28-1.76]). Nuclear Ep-ICD overexpression was significantly associated with early tumor grade (Grade I and II) (53 of 108 patients, 49%; p=0.018, OR=0.46, 95% CI=0.24-0.89) and no lymph node metastases at surgery (58 of 123 patients, 47%; p=0.028, OR=0.48, 95% CI=0.24-0.98). Cytoplasmic Ep-ICD accumulation was also observed in all but one patient with clinical recurrence (24 of 25 patients, 96%; p=0.035, OR=6.75, 95% CI=0.88-51.67). No association was observed between nuclear or cytoplasmic Ep-ICD and ER/PR status, AJCC pTNM stage, T-stage, tumor size, or patient's age at diagnosis (Table 3). Membranous EpEx or loss of membranous EpEx did not show significant correlation with any of the clinico-pathological parameters in this cohort of breast cancer patients (data not shown).

TABLE 3
Nuclear and cytoplasmic Ep-ICD expression in invasive ductal
carcinoma (IDC) and correlation with clinicopathological parameters (“a” indicates that tumor
size was available for only 177 of 180 IDC cases; “b” indicates that tumor grades were
available for only 173 of 180 IDC cases; “c” indicates that ER and PR status was available
for only 169 and 167 of 180 IDCs cases, respectively).

| | Total | Ep-ICD | | | | Ep-ICD | | | |
|---|---|---|---|---|---|---|---|---|---|
| Clinicopathological | Cases | Nuclear | | p- | Odd's ratio | Cytoplasm | | p- | Odd's ratio |
| parameters | (n = 180) | n | (%) | value | (95% C.I.) | n | (%) | value | (95% C.I.) |
| IDC cases | | 75 | 42 | — | — | 145 | 81 | — | — |
| Age | | | | | | | | | |
| <59 yrs | 88 | 39 | 44.3 | | | 74 | 84.1 | | |
| ≧59 yrs | 92 | 36 | 39.1 | 0.480 | 0.80 (0.45-1.45) | 71 | 77.2 | 0.241 | 0.64 (0.30-1.36) |
| Tumor Size^{a} | | | | | | | | | |
| ≦2 cm | 81 | 35 | 43.2 | | | 69 | 85.2 | | |
| >2 cm | 96 | 37 | 38.5 | 0.529 | 0.82 (0.45-1.50) | 73 | 76.0 | 0.128 | 0.55 (0.25-1.20) |
| T-stage | | | | | | | | | |
| T₁ + T₂ | 171 | 71 | 41.5 | | | 138 | 80.7 | | |
| T₃ + T₄ | 9 | 4 | 44.4 | 0.862 | 1.13 (0.30-4.34) | 7 | 77.8 | 0.829 | 0.84 (0.17-4.22) |
| Nodal Status | | | | | | | | | |
| Nₓ₊₀ | 123 | 58 | 47.2 | | | 99 | 80.5 | | |
| N₁₋₃ | 57 | 17 | 29.8 | 0.028 | 0.48 (0.24-0.98) | 46 | 80.7 | 0.973 | 1.02 (0.45-2.24) |
| Stage | | | | | | | | | |
| I + II | 159 | 68 | 42.8 | | | 130 | 81.8 | | |
| III + IV | 21 | 7 | 33.3 | 0.410 | 0.67 (0.26-1.74) | 15 | 71.4 | 0.261 | 0.56 (0.20-1.56) |
| Grade^{b} | | | | | | | | | |
| I + II | 108 | 53 | 49.1 | | | 90 | 83.3 | | |
| III | 65 | 20 | 30.8 | 0.018 | 0.46 (0.24-0.89) | 48 | 73.8 | 0.132 | 0.57 (0.27-1.20) |
| Clinical | | | | | | | | | |
| Recurrence | | | | | | | | | |
| No | 155 | 50 | 32.3 | | | 121 | 78.1 | | |
| Yes | 25 | 25 | 100 | <0.001 | 1.50 (1.28-1.76) | 24 | 96.0 | 0.035 | 6.75 (0.88-51.67) |
| ER/PR status^{c} | | | | | | | | | |
| ER⁺ | 136 | 62 | 45.6 | | | 112 | 82.4 | | |
| ER⁻ | 33 | 12 | 36.4 | 0.338 | 1.47 (0.67-3.22) | 25 | 75.8 | 0.386 | 1.49 (0.60-3.71) |
| PR⁺ | 103 | 49 | 47.6 | | | 88 | 85.4 | | |
| PR⁻ | 64 | 25 | 39.1 | 0.282 | 1.42 (0.75-2.67) | 48 | 75.0 | 0.092 | 1.96 (0.89-4.30) |
| ER⁺PR⁺ | 103 | 49 | 47.6 | | | 88 | 85.4 | | |
| ER⁻PR⁻ | 33 | 12 | 36.4 | 0.260 | 1.59 (0.70-3.56) | 25 | 75.8 | 0.197 | 1.96 (0.89-4.30) |

Occurrence of an adverse clinical event (recurrence, distal metastases, and/or death) among all breast carcinoma patients was observed in 42 of 121 (34.7%) patients. Subgroup analysis of IDC patients alone that were positive for nuclear Ep-ICD showed an adverse clinical event in 25 of 75 (33.3%) patients. In the entire cohort of breast carcinoma patients, only patients who were positive for nuclear Ep-ICD accumulation had adverse clinical events. Evaluation of all patients who had experienced an adverse clinical event or recurrence showed that of these 42 patients, 37 (88.1%) had early stage tumors (AJCC pTNM Stage I or II), while 5 (11.9%) were Stage III or IV tumors. Among the 25 IDC patients who had adverse clinical events, 21 of 25 (84%) had early stage tumors (AJCC pTNM Stage I and II), while 4 of 25 (16%) were AJCC pTNM Stage III and IV cases.

Prognostic Use of Ep-ICD Expression for Disease-Free Survival

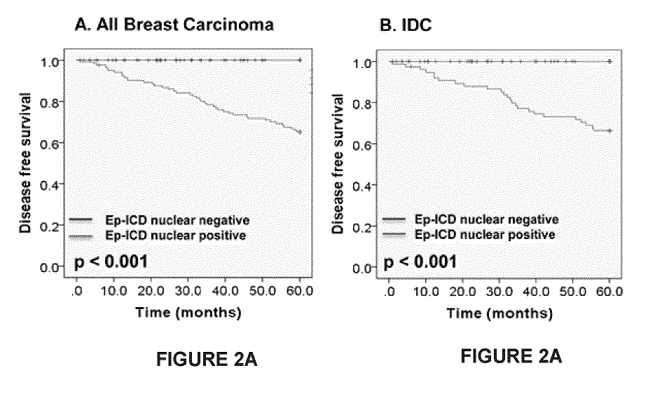

The association between nuclear Ep-ICD accumulation, clinicopathological parameters and disease-free survival was evaluated (Table 4). Significant association was observed between nuclear Ep-ICD expression and disease-free survival (p<0.001), with a decreased third quartile survival time of 40.9 months (FIG. 2A). In contrast, all patients who did not show nuclear Ep-ICD positivity were alive and free of disease even after 5-years post-treatment. Cox multivariate regression analysis identified nuclear Ep-ICD as the most important prognostic marker for an adverse clinical event (p=0.008, Hazard Ratio (HR)=70.47, 95% C.I.=3.00-1656.24; Table 4). Subgroup analysis of IDC patients also showed significant association between nuclear Ep-ICD expression and disease-free survival (p<0.001) with a decreased third quartile survival time of 39.5 months (FIG. 2B). In contrast, all patients with no nuclear Ep-ICD positivity were alive and free of disease as of 5-years following surgery. Among the IDC cases, Cox multivariate regression analysis showed nuclear Ep-ICD to be the most important prognostic marker for an adverse clinical event (p=0.011, HR=80.18, 95% C.I.=2.73-2352.2). Fifty of the 75 nuclear Ep-ICD positive IDC patients did not have recurrence during the 5-year follow up period.

TABLE 4
Kaplan-Meier Survival Analysis And Multivariate Cox Regression
Analysis For Breast Cancer Patients

| | Kaplan- | Multivariate | | |
|---|---|---|---|---|
| | Meier | Cox | | |
| | survival | regression | | |
| | analysis | analysis | Hazard's | |
| | unadjusted | adjusted | Ratio | |
| | P-value | P-value | (H.R.) | 95% C.I. |
| All Breast | | | | |
| Carcinomas | | | | |
| Nuclear | <0.001 | 0.008 | 70.47 | 3.00-1656.24 |
| Ep-ICD⁺ | | | | |
| Cytoplasmic | 0.115 | 0.860 | — | — |
| Ep-ICD⁺ | | | | |
| Age | 0.081 | 0.178 | — | — |
| Tumor size | 0.676 | 0.518 | — | — |
| T-stage | 0.315 | 0.388 | — | — |
| Nodal status | 0.963 | 0.190 | — | — |
| Clinical Stage | 0.064 | 0.260 | — | — |
| Grade | 0.094 | 0.035 | — | — |
| ER status | 0.292 | 0.654 | — | — |
| PR status | 0.827 | 0.790 | — | — |
| IDC Tumors | | | | |
| Nuclear | <0.001 | 0.011 | 80.183 | 2.733-2352.2 |
| Ep-ICD⁺ | | | | |
| Cytoplasmic | 0.048 | 0.496 | — | — |
| Ep-ICD⁺ | | | | |
| Age | 0.796 | 0.787 | — | — |
| Tumor size | 0.556 | 0.516 | — | — |
| T-stage | 0.237 | 0.366 | — | — |
| Nodal status | 0.814 | 0.398 | — | — |
| Clinical Stage | 0.129 | 0.809 | — | — |
| Grade | 0.329 | 0.062 | — | — |
| ER status | 0.384 | 0.678 | — | — |
| PR status | 0.984 | 0.499 | — | — |

Nuclear Ep-ICD was more frequently expressed in breast cancers as compared to normal tissues. Significant association was observed between increased nuclear Ep-ICD expression and reduced disease-free survival in patients with ductal carcinoma in situ (DCIS) and invasive ductal carcinoma (IDC) (p<0.001). Nuclear Ep-ICD was positive in all the 13 DCIS and 25 IDC patients who had reduced disease-free survival, while none of the nuclear Ep-ICD negative DCIS or IDC patients had recurrence during the follow up period. Notably, majority of IDC patients who had recurrence had early stage tumors. Multivariate Cox regression analysis identified nuclear Ep-ICD as the most significant predictive factor for reduced disease-free survival in IDC patients (p=0.011, Hazard ratio=80.18).

ESLI Results

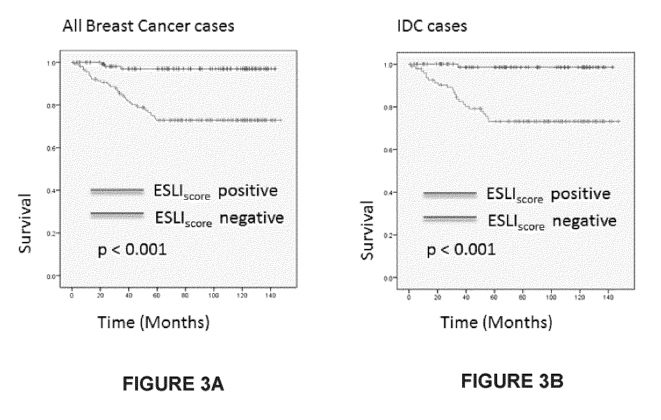

A significant association was observed between ESLI values of ≧3 and reduced disease-free survival in all breast cancer patients (p<0.001; FIG. 3A); median survival for ESLI positive cases (i.e., ESLI values of ≧3) was 139.3 months and ESLI negative cases (i.e., ESLI values of ≦3) was 115.5 months. A significant association was observed between ESLI values of ≧3 and reduced disease-free survival in invasive ductal carcinoma (IDC) patients (p<0.001; FIG. 3B); median survival for ESLI positive cases was 141.3 months and ESLI negative cases was 115.5 months (p<0.001).

Discussion

As mentioned above, the inventors previously reported nuclear and cytoplasmic Ep-ICD expression in ten different epithelial cancers, including breast cancers (Ralhan et al., BMC Cancer 2010; US Patent Publication No. 2011/0275530). However, the previous report did not examine the correlation of nuclear Ep-ICD expression with clinical parameters or its prognostic utility in the ten epithelial cancers, including breast cancer. The current study assessed the suitability of Ep-ICD as a marker for predicting prognosis of breast cancer. Although expression of the full length EpCAM protein has been widely investigated in human malignancies, the expression and subcellular localization of its intracellular domain, Ep-ICD, has not been well-characterized in clinical specimens. The present data indicate that there are significant differences in Ep-ICD expression in normal relative to malignant breast tissues and in non-aggressive relative to aggressive breast cancers.

In the present study, high occurrence of disease recurrence, distal metastases, and/or death was observed among IDC patients positive for nuclear Ep-ICD post-therapeutic treatment. In contrast, no recurrence distal metastases, or death was observed in nuclear Ep-ICD negative patients during a 5-year follow up period post-therapeutic treatment. The majority of patients with disease recurrence (37 of 42, 88.1%) had early stage breast carcinomas (AJCC pTNM Stage I and II) that would normally be considered lower-risk for future recurrence. No nuclear Ep-ICD negative patient suffered disease recurrence. These observations support the finding that nuclear Ep-ICD presence and accumulation, even in early stage breast tumors, can be used to predict aggressive breast cancer.

The presence of nuclear Ep-ICD, irrespective of tumor stage or any other clinical variable predicted a high risk of disease recurrence within a 5-year period post-therapeutic treatment. Multivariate Cox regression analyses identified nuclear Ep-ICD accumulation as the most significant factor for prediction of recurrence in IDC patients.

### CONCLUSIONS

Patients with nuclear Ep-ICD positive breast tissues post-therapeutic treatment had poor prognosis relative to patients having breast tissues lacking nuclear Ep-ICD. The high recurrence of disease in nuclear Ep-ICD positive patients, especially those with early tumor stage suggests that nuclear Ep-ICD presence and accumulation may be used to identify aggressive breast cancers, including early stage aggressive breast cancers, which would likely benefit from more rigorous post-operative surveillance and/or treatment. The ESLI algorithm developed by the present inventors provides a unique tool for use in breast cancer prognosis.

Although the invention has been described with reference to certain specific embodiments, various modifications thereof will be apparent to those skilled in the art. Any examples provided herein are included solely for the purpose of illustrating the invention and are not intended to limit the invention in any way. Any drawings provided herein are solely for the purpose of illustrating various aspects of the invention and are not intended to be drawn to scale or to limit the invention in any way. The scope of the claims appended hereto should not be limited by the preferred embodiments set forth in the above description, but should be given the broadest interpretation consistent with the present specification as a whole. The disclosures of all prior art recited herein are incorporated herein by reference in their entirety.

We claim:

1. A method for prognosing breast cancer in a subject, the method comprising:
(a) measuring an amount of nuclear Ep-ICD in a biological sample from the subject;
(b) comparing the amount measured in the biological sample to a control; and
(c) prognosing breast cancer based on the comparison between the measured amount of nuclear Ep-ICD and the control.

2. The method of claim 1, wherein if the control is:
an amount of nuclear Ep-ICD in a non-aggressive breast cancer sample, then a higher measured amount of nuclear Ep-ICD indicates a poor prognosis, and an equal or lower measured amount of nuclear Ep-ICD indicates a favorable prognosis; or
an amount of nuclear Ep-ICD in an aggressive breast cancer sample, then an equal or higher measured amount of nuclear Ep-ICD indicates a poor prognosis.

3. The method of claim 2, wherein the non-aggressive breast cancer sample is known not to progress in disease for at least 40 months following measurement of the nuclear Ep-ICD amount.

4. The method of claim 2 or 3, wherein the aggressive breast cancer sample is known to progress in disease in less than about five years following measurement of the nuclear Ep-ICD amount.

5. The method of any one of claims 2 to 4, wherein the poor prognosis comprises disease free survival of less than five years.

6. The method of claim 5, wherein the disease free survival is less than or equal to about 41 months.

7. The method of any one of claims 2 to 6, wherein the favorable prognosis comprises disease free survival of at least about five years.

8. The method of any one of claims 1 to 7, wherein the biological sample from the subject is obtained post-therapeutic treatment.

9. The method of any one of claims 1 to 8, wherein the biological sample from the subject comprises one or more of breast epithelial cells, breast tissue, breast tumor tissue, and stage I or II breast cancer tumor cells.

10. The method of any one of claims 1 to 9, wherein the breast cancer prognosed is invasive ductal carcinoma, invasive lobular carcinoma, invasive mucinous carcinoma, ductal carcinoma in situ, or lobular carcinoma in situ.

11. The method of any one of claims 1 to 10, wherein the measured amount of nuclear Ep-ICD is one or more of a quantitative and qualitative amount.

12. The method of claim 11, wherein the quantitative amount is a percentage of cells in the biological sample that are positive for nuclear Ep-ICD or an absolute quantity of nuclear Ep-ICD.

13. The method of claim 11 or 12, wherein the qualitative amount is an intensity of signal emitted by a label indicative of nuclear Ep-ICD.

14. The method of claim 13, further comprising determining quantitative and qualitative scores for nuclear Ep-ICD and cytoplasmic Ep-ICD, wherein increased quantitative and qualitative nuclear and cytoplasmic Ep-ICD scores are associated with a poor prognosis of breast cancer.

15. The method of claim 14, wherein the determining of the quantitative and qualitative nuclear Ep-ICD and cytoplasmic Ep-ICD scores comprises:
(i) contacting the sample with: a binding agent that specifically binds to Ep-ICD or part thereof and a detectable label for detecting binding of the first binding agent to Ep-ICD, wherein the detectable label emits a detectable signal upon binding of the binding agent to Ep-ICD;
(ii) measuring:
(a) a first percentage, comprising the percentage of cells in the sample having Ep-ICD in the nucleus bound to the binding agent, and assigning a first quantitative score to the first percentage according to a first scale;
(b) a second percentage, comprising the percentage of cells in the sample having Ep-ICD in the cytoplasm bound to the binding agent, and assigning a second quantitative score to the second percentage according to the first scale;
(iii) measuring:
(a) a first intensity, comprising the intensity of the signal emitted in the nucleus by the label, and assigning a first qualitative score to the first intensity according to a second scale;
(b) a second intensity, comprising the intensity of the signal emitted in the cytoplasm by the label and assigning a second qualitative score to the second intensity according to the second scale.

16. The method of claim 15, further comprising calculating total nuclear Ep-ICD and cytoplasmic Ep-ICD scores, the calculating comprising
(a) adding the first quantitative and qualitative scores to generate the total nuclear Ep-ICD score;
(b) adding the second quantitative and qualitative scores to generate the total cytoplasmic Ep-ICD score.

17. The method of claim 16 further comprising:
calculating an Ep-ICD Subcellular Localization Index (ESLI) value for the sample, the ESLI value being a sum of the total nuclear Ep-ICD score and the total cytoplasmic Ep-ICD score, divided by two;
comparing the calculated ESLI value to a reference value, wherein the reference value is:
(i) an ESLI value indicative of a non-aggressive breast cancer; or
(ii) an ESLI value indicative of an aggressive breast cancer; and
determining a poor prognosis of breast cancer in the subject when the calculated ESLI value of the sample is greater than the reference value of (i) or is greater than or equal to the reference value of (ii).

18. The method of any one of claims 15 to 17, wherein the binding agent is an antibody.

19. The method of any one of claims 15 to 18, wherein the label is chosen from detectable radioisotopes, luminescent compounds, fluorescent compounds, enzymatic labels, biotinyl groups and predetermined polypeptide epitopes recognizable by a secondary reporter.

20. The method of any one of claims 11 to 19, wherein the quantitative amount is obtained using immunohistochemical (IHC) analysis.

21. The method of any one of claims 11 to 20, wherein the qualitative amount is obtained using immunohistochemical (IHC) analysis.

22. The method of any one of claims 15 to 21, wherein the first scale comprises the following scores:
a score of 0 is assigned when less than 10% of the cells are positive;
a score of 1 is assigned when 10-30% of the cells are positive;
a score of 2 is assigned when 31-50% the cells are positive;
a score of 3 is assigned when 51-70% of the cells are positive; and
a score of 4 is assigned when more than 70% of the cells are positive, and wherein the second scale comprises the following scores:
a score of 0 is assigned when no signal is detected;
a score of 1 is assigned when a mild signal is detected;
a score of 2 is assigned when a moderate signal is detected; and
a score of 3 is assigned when an intense signal is detected.

23. The method of any one of claims 17 to 22, wherein an ESLI value indicative of non-aggressive breast cancer is less than 3 and an ESLI value indicative of aggressive breast cancer is greater than or equal to 3.

24. The method of any one of claims 1 to 23, wherein the measuring of an amount of nuclear Ep-ICD is manual or automated.
